(19) United States
(12) Patent Application Publication
(10) Pub. No.: US 2020/0333409 A1
(43) Pub. Date: Oct. 22, 2020

(54) MAGNETIC REFERENCE SENSOR WITH REDUCED SENSITIVITY TO MAGNETIC DISTORTIONS

(71) Applicant: ST. JUDE MEDICAL, CARDIOLOGY DIVISION, INC., St. Paul, MN (US)

(72) Inventors: Alexander P. Thome, St. Paul, MN (US); Chad T. Glader, Ramsey, MN (US); Scott Meyerson, Ham Lake, MN (US)

(21) Appl. No.: 16/850,955

(22) Filed: Apr. 16, 2020

Related U.S. Application Data
(60) Provisional application No. 62/836,434, filed on Apr. 19, 2019.

(51) Int. Cl. G01R 33/20; A61B 5/055; A61B 5/08

(52) U.S. Cl. CPC G01R 33/20; A61B 5/0816; A61B 5/055

(57) ABSTRACT

Aspects of the present disclosure are directed to systems, apparatuses, and methods for detecting and correcting for patient respiration within a medical magnetic localization system. In one embodiment of the present disclosure, a system is disclosed for detecting patient respiration in a magnetic field for localization of an intravascular catheter. The system including a magnetic field generator and a magnetic reference sensor. The magnetic field generator generates the magnetic field for localization of the catheter within the patient. The magnetic reference sensor includes sensor coils that have a longitudinal axis, sense the magnetic field aligned with the orientation of the sensor coil, and output an electrical signal indicative of the sensed magnetic field. The magnetic reference sensor is positioned within the magnetic field generated by the magnetic field generator, and the longitudinal axis of each sensor coil is non-parallel relative to an axis of the generated magnetic field.

### CROSS-REFERENCE TO RELATED APPLICATION

This application claims the benefit of U.S. application No. 62/836,434, filed 19 Apr. 2019, which is hereby incorporated by reference as though fully set forth herein.

### BACKGROUND

### a. Field

The present disclosure relates generally to the magnetic localization of medical instruments within a human body. More specifically, the instant disclosure relates to the use of magnetic reference sensors within a magnetic localization system to track patient respiration.

### b. Background Art

Electrophysiology (EP) catheters have been used for an ever-growing number of procedures. For example, catheters have been used for diagnostic, therapeutic, mapping and ablative procedures, to name just a few examples. Typically, a catheter is manipulated through the patient's vasculature to the intended site, for example, a site within the patient's heart, and carries one or more electrodes, which may be used for diagnosis, mapping, ablation, or other treatments. Precise positioning of the catheter and clinician knowledge of the precise location within the body of the patient is desirable for improved procedure success rates.

To precisely position a catheter within the body at a desired site, some type of localization must be used. To determine the relative position of the catheter to patient anatomy, magnetic localization systems have been developed that provide a location of the catheter within a well-known and controlled magnetic field. The externally-generated magnetic fields include precise magnetic gradients (field lines) that are sensed by the catheter (e.g., sensing coils) being located within the magnetic field. The currents induced by the magnetic field(s) on the sensing coils are analyzed using algorithmic processes and used to determine the position of the catheter within the patient's body. Once the catheter is positioned within the patient, as desired, a clinician may operate the catheter, for example, to ablate tissue to interrupt potentially pathogenic heart rhythms. Magnetic reference sensors are commonly used in conjunction with localization systems to track and compensate for patient respiration. It has been discovered that magnetic reference sensors are susceptible to error induced by magnetic distortions within the magnetic field caused by, for example, extraneous ferrous or metallic objects intruding into the magnetic field. The introduction of such distortions may result in the system presenting an inaccurate position of the catheter within the patient's body, and limit the efficacy of a medical procedure.

The foregoing discussion is intended only as an exemplary illustration of the present field and is not intended to limit the claim scope.

### BRIEF SUMMARY

Various embodiments of the present disclosure reduce sensitivity of magnetic reference sensors within a magnetic localization system to magnetic distortions associated with the intrusion of metallic objects in a magnetic field used for localization of a medical device.

Aspects of the present disclosure are directed to a system for detecting patient respiration in a magnetic field for localization of an intravascular catheter within a patient. The system including a magnetic field generator and a magnetic reference sensor. The magnetic field generator generates the magnetic field for localization of the catheter within the patient. The magnetic reference sensor includes more than one sensor coil. Each of the sensor coils have a longitudinal axis, sense the magnetic field aligned with the orientation of the sensor coil, and output an electrical signal indicative of the sensed magnetic field. The magnetic reference sensor is positioned within the magnetic field generated by the magnetic field generator, and the longitudinal axis of each sensor coil is non-parallel relative to an axis of the generated magnetic field. In some specific embodiments, the longitudinal axis of each of the sensor coils are positioned substantially orthogonal relative to the axis of the generated magnetic field.

Some embodiments of the present disclosure are directed to an apparatus for detecting a position within a magnetic field. The apparatus includes a first sensor coil, a second sensor coil oriented orthogonal to the first sensor coil and fixedly positioned relative to the first sensor coil, and a third sensor coil oriented orthogonal to the first and second sensor coils and fixedly positioned relative to the first and second sensor coils. The first, second, and third sensor coils sense a magnetic field strength of the magnetic field oriented substantially coaxial with the respective sensor coil, and each of the sensor coils are positioned with substantially equal perpendicularity relative to a magnetic field axis. In specific embodiments, each of the sensor coils include proximal and distal ends, each of the proximal ends of the sensors coils lie in a first common plane, and each of the distal ends of the sensors coils lie in a second common plane. In addition, each of the sensor coils may have a non-vertical orientation.

Additional features, advantages, and embodiments of the disclosure may be set forth or apparent from consideration of the detailed description and drawings. Moreover, it is to be understood that the foregoing brief summary and the following detailed description, drawings, and attachment are exemplary and intended to provide further explanation without limiting the scope of the disclosure as claimed.

### BRIEF DESCRIPTION OF THE DRAWINGS

The accompanying drawings, which are included to provide a further understanding of the disclosure, are incorporated in and constitute a part of this specification, illustrate embodiments of the disclosure and together with the detailed description serve to explain the principles of the disclosure.

FIG. 1 is a schematic diagram of a magnetic localization system, consistent with various aspects of the present disclosure.

FIG. 2A is a fragmentary, isometric view of a catheter assembly comprising a catheter configured for localization in a magnetic localization system and an introducer, consistent with various aspects of the present disclosure.

FIG. 2B is an enlarged, fragmentary side view of the distal tip assembly of the catheter of FIG. 2A, consistent with various aspects of the present disclosure.

FIG. 3A is an isometric top view of a magnetic reference sensor housing, consistent with various aspects of the present disclosure, with portions of the sensor housing broken away to reveal internal features.

FIG. 3B is an isometric top view of a magnetic reference sensor including three sensor coils, consistent with various aspects of the present disclosure, with portions of the sensor housing broken away to reveal internal features.

FIG. 3C is a close-up, isometric top view of the magnetic reference sensor of FIG. 3B including three sensor coils, sans the sensor housing, consistent with various aspects of the present disclosure.

FIG. 3D is a close-up, isometric side view of the magnetic reference sensor of FIG. 3B, sans the magnetic detection sensor housing, and illustrating the perpendicularity of sensor coils of the magnetic reference senor, consistent with various aspects of the present disclosure.

FIG. 3E is a top view of the three sensor coils of the magnetic reference sensor of FIG. 3B, consistent with various aspects of the present disclosure.

FIG. 3F is a side view of the three sensor coils of the magnetic reference sensor of FIG. 3B, consistent with various aspects of the present disclosure.

FIG. 4 is a side view of an example application of a magnetic reference sensor and a magnetic detection sensor during a medical procedure, consistent with various aspects of the present disclosure.

While various embodiments discussed herein are amenable to modifications and alternative forms, aspects thereof have been shown by way of example in the drawings and will be described in further detail. It should be understood, however, that the intention is not to limit the disclosure to the particular embodiments described. On the contrary, the intention is to cover all modifications, equivalents, and alternatives falling within the scope of the disclosure including aspects defined in the claims.

### DETAILED DESCRIPTION

Cardiac localization systems are capable of displaying a three-dimensional (3D) position of conventional electrophysiology catheters within an overlaid model or image of a cardiac chamber, for example. These localization systems may also display cardiac electrical activity as waveform traces and as dynamic 3-D isopotential maps on the model of the cardiac chamber. The contoured surfaces of these three dimensional models may be based on the anatomy of the patient's own cardiac chamber. These localization systems may use impedance based and/or magnetic based localization technologies to render catheter position, facilitate model creation, and navigation therethrough.

When using magnetic localization, the magnetic field(s) generated from a local source are inherently susceptible to distortions caused by metallic or ferrous objects intruding into, or being placed adjacent to, the generated magnetic field(s). Such distortions can cause inaccuracies in calculated or determined medical device locations, and in related anatomical models and other representations. These distortions may also impede the efficacy of ancillary sensors such as magnetic reference sensors which track patient respiration.

Magnetic sensors embedded within intracardiac catheters are used to determine position and orientation of the catheter with respect to one or more known reference positions. This magnetic position and orientation information can be used to navigate the catheter and can also be used to optimize impedance-based catheter localization (when the localization system is a hybrid-type system). When navigating a catheter through a magnetic field, the displayed or otherwise reported positions of the catheters can notably shift (e.g., visually shift on a screen displaying a representation of the catheter location) when the underlying magnetic field is changed/distorted despite no actual change (or minimal actual change) in the catheter's physical location. Understandably, this shift can cause inaccuracies to models created using the reported locations of the catheters, reported ablation locations, etc. With regard to patient reference sensors, distortion related inaccuracy may cause the system to ignore a signal output from the patient reference sensor entirely. Embodiments of the present disclosure, as described in more detail below with reference to the figures, reduce the sensitivity of magnetic reference sensors to such distortions within a magnetic field.

In magnetic-field based localization systems, a magnetic reference sensor may be tracked positionally within a generated magnetic field. The sensor coils of the magnetic reference sensor are sensitive to instability and inaccuracies associated with weakened magnetic field signals in certain regions of the generated magnetic field and distortions in the magnetic field due to the ingress of ferrous materials. In various embodiments of the present disclosure, a magnetic reference sensor is disclosed including a novel out-of-plane configuration of three magnetic sensor coils which reduces the sensitivity of the navigation system to distortions in the magnetic field and increases the robustness of the system in such compromised environments. The magnetic reference sensor may include three (ferric core) sensor coils that are oriented perpendicular relative to one another. In one specific embodiment, the sensors coils of the magnetic reference sensor are secured within a housing in such a way that proximal ends of each of the sensor coils lie in a first common plane, and distal ends of each of the sensor coils lie in a second common plane. To reduce sensitivity to magnetic distortions, especially those in proximity to a magnetic field emitter, the sensor coils of the magnetic reference sensor are positioned such that a longitudinal axis of each sensor coil is out of alignment with the magnetic field emitter. It has been discovered that prior art magnetic reference sensors in magnetic-field based navigation systems suffer from orientation specific error associated with alignment of one of the sensor coils and the magnetic field generator. Specifically, where one of the three sensor coils is aligned with the magnetic field generator, in response to a magnetic-field distortion, the navigation system may nullify the data signal from the generator-aligned sensor coil or the entire magnetic reference sensor. Moreover, in such an event, the localization system may be unable to compensate for patient respiration. As a result, embodiments described herein utilize a sensor coil configuration wherein all of the sensor coils are equally out of alignment with a magnetic field transmitter.

In some systems such as a fluoroscopic imaging system in which the magnetic field emitter is attached to a C-arm of a fluoroscopic imager, the presence of the imager can, in some circumstances, cause distortion to the resulting magnetic field generated by the magnetic field emitter of a magnetic-based localization system. Aspects of the present disclosure are directed to identifying the distortion and correcting for it in the calculated position of one or more sensors within the magnetic field.

Another benefit of the present disclosure is that the configuration of the sensor coils in the magnetic reference sensor also allows the magnetic localization system to operate in magnetic fields with large magnetic anomalies (e.g., magnetically caustic environments) as all three sensor coil inputs to the localization system are maintained.

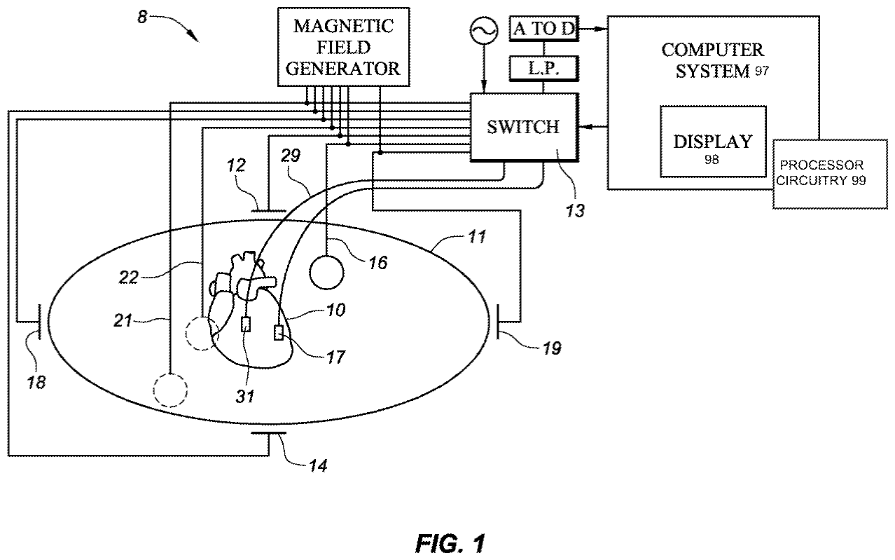

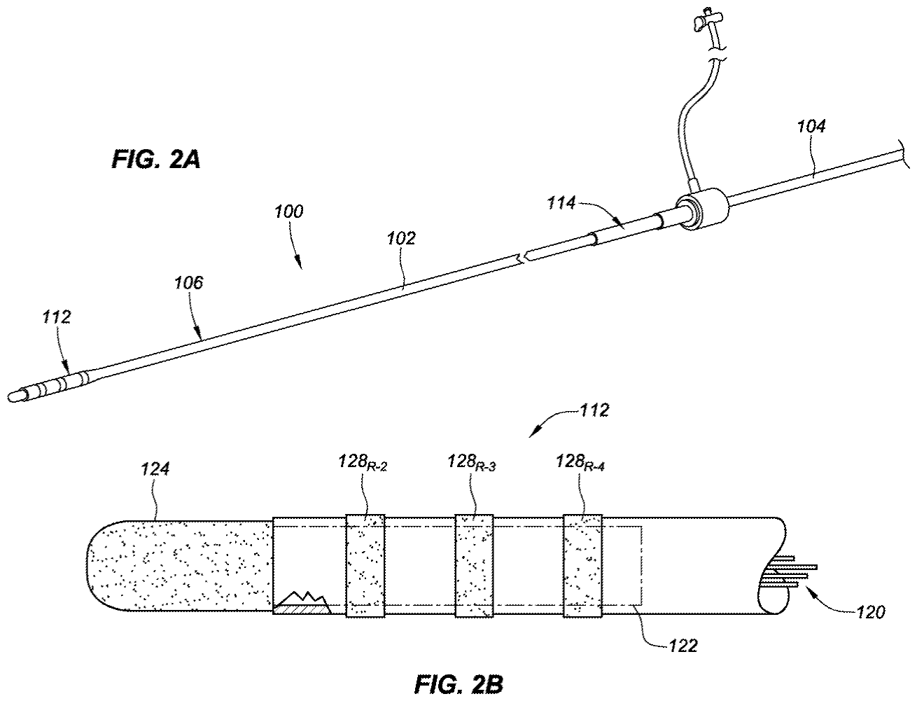

FIG. 1 shows a schematic diagram of a magnetic localization system 8 used for navigating the human anatomy of a patient 11 (depicted, for simplicity's sake, as an oval in FIG. 1) while conducting a medical procedure. For example, as shown in FIG. 1, the system 8 may be used to map a heart 10 of the patient and to navigate a cardiac catheter through the chambers of the heart. Magnetic localization system 8 determines the location (and, in some embodiments, the orientation) of objects (e.g., a portion of a diagnostic or ablation catheter, such as the electrode assembly 112 depicted in FIGS. 2A and 2B), typically within a three-dimensional space, and expresses those locations as position information determined relative to at least one reference. Specifically, the magnetic localization system 8 may be used to determine the location of the cardiac catheter within a magnetic field, which is then overlaid onto, for example, an image or a model of the heart 10. In other embodiments, magnetic resonance imaging data, among other reference data may be overlaid onto the three-dimensional space to provide a clinician with a virtual work environment in which to reference for real-time position of the cardiac catheter relative to the patient's heart 10.

The magnetic localization system 8 may include various visualization, localization, mapping, and navigation components. For example, the localization system 8 may comprise a magnetic-field-based system such as the CARTO™ system commercially available from Biosense Webster, and as generally shown with reference to one or more of U.S. Pat. Nos. 6,498,944; 6,788,967; and 6,690,963, the disclosures of which are hereby incorporated by reference in their entireties as though fully set forth herein. In another exemplary embodiment, the localization system 8 may comprise a magnetic field based system such as the MEDIGUIDE™ Technology system available from St. Jude Medical, Inc., and as generally shown with reference to one or more of U.S. Pat. Nos. 6,233,476; 7,197,354; 7,386,339; U.S. patent application Ser. No. 14/208,120 entitled “Medical Device Navigation System” filed on 13 Mar. 2014, U.S. Provisional Patent Application No. 61/834,223 entitled “Medical Device Navigation System” filed on 12 Jun. 2013, and International Application No. PCT/IB2014/059709 entitled “Medical Device Navigation System” filed on 13 Mar. 2014, the disclosures of which are hereby incorporated by reference in their entireties as though fully set forth herein. In yet another embodiment, the localization system 8 may comprise a hybrid-type system (i.e., electric-field-based and magnetic-field-based system), such as, for example and without limitation, the EnSite Precision™ system from St. Jude Medical systems, the systems described in pending U.S. patent application Ser. No. 13/231,284 entitled “Catheter Navigation Using Impedance and Magnetic Field Measurements” filed on 13 Sep. 2011 and U.S. patent application Ser. No. 13/087,203 entitled “System and Method for Registration of Multiple Navigation Systems to a Common Coordinate Frame” filed on 14 Apr. 2011, each of which is hereby incorporated by reference in its entirety as though set fully forth herein, or the CARTO™ 3 system commercially available from Biosense Webster. In yet still other exemplary embodiments, the localization system 8 may comprise or be used in conjunction with other commonly available systems, such as, for example and without limitation, fluoroscopic, computed tomography (CT), and magnetic resonance imaging (MRI) based systems. For purposes of clarity and illustration only, the localization system 8 will be described hereinafter as comprising a magnetic field based localization system.

Embodiments of the present disclosure may include various visualization, mapping and navigation components as known in the art, including, for example, the EnSite Precision™ commercially available from St. Jude Medical, Inc., or as seen generally by reference to U.S. Pat. No. 7,263,397 (the '397 patent), or U.S. Patent Publication No. 2007/0060833 A1, U.S. application Ser. No. 11/227,580 filed 15 Sep. 2005 (the '580 application). The '397 patent and the '580 application are both hereby incorporated by reference as though fully set forth herein.

FIG. 1 may further exemplify a hybrid-type localization system including both an impedance-based localization system and a magnetic-based localization system.

In general, and as shown in FIG. 1, localization system 8 includes one or more magnetic field transmitters (e.g., one or more of 12, 14, 16, 18, 19, and 21) that emit a magnetic field across the patient's body 11. These magnetic field transmitters, which may be placed upon or attached/applied to the patient, or fixed to an external apparatus, define three generally orthogonal axes, e.g., an x-axis, a y-axis, and a z-axis. The magnetic field transmitters are electrically coupled to a magnetic field generator. The magnetic field generator generates one or more magnetic fields that may be transmitted simultaneously, time multiplexed, and/or frequency multiplexed via the one or more magnetic field transmitters. A switch 13 samples the signals received from one or more receivers (e.g., one or more of 17, 22, and 31; a catheter, a patient reference sensor (or magnetic reference sensor), an internal reference sensor, a metal distortion sensor, etc.). The received signals from the receivers, indicative of the magnetic field that traversed through the patient's body 11, are then converted from an analog to a digital signal for further processing by the computer system 97. In addition to display 98, the computer system includes processor circuitry 99. In various embodiments, the processor circuitry 99 may include hardware/software (modules) for conducting magnetic field detection, distortion identification, and position correction. The computer system performs computations on the data received from the receivers to determine, for example, the location of a cardiac catheter within the patient's heart. However, the actual catheter position may be obscured by magnetic distortions within the magnetic field caused by other ferrous/metallic bodies. These magnetic distortions are associated with an error rate of the perceived position of the catheter compared to the actual position of the catheter.

For reference by a clinician during a procedure, the perceived location of the catheter within the magnetic field can be presented on a display 98 in relation to known reference points, e.g., cardiac chambers, arteries, etc.

For purposes of this disclosure, an example medical device, such as a catheter may extend into the left ventricle of the patient's heart 10. The catheter includes a plurality of sensor coils spaced along its length. As used herein, the term “sensor coils” generically refer to any element whose position within a magnetic field can be measured by that system (e.g., magnetic sensors). Because each sensor coil lies within the magnetic field, localization data may be collected simultaneously for each sensor coil.

A magnetic-based localization system 8 may include a fixed reference 22 to define the origin of the magnetic-based localization system's coordinate frame. This fixed reference provides a relative position to which the positions of sensor coils on the catheter are measured. Such a fixed reference can likewise be in a fixed internal or external location. Likewise, multiple references may be used for the same or different purposes (e.g., to correct for respiration, patient shift, system drift, or the like).

A computer system 97, which can comprise a conventional general-purpose computer, a special-purpose computer, a distributed computer, or any other type of computer, and which can comprise one or more processors, such as a single central processing unit (CPU), or a plurality of processing units, commonly referred to as a parallel processing environment, may control magnetic localization system 8 and/or execute instructions to practice the various aspects of the embodiments described herein.

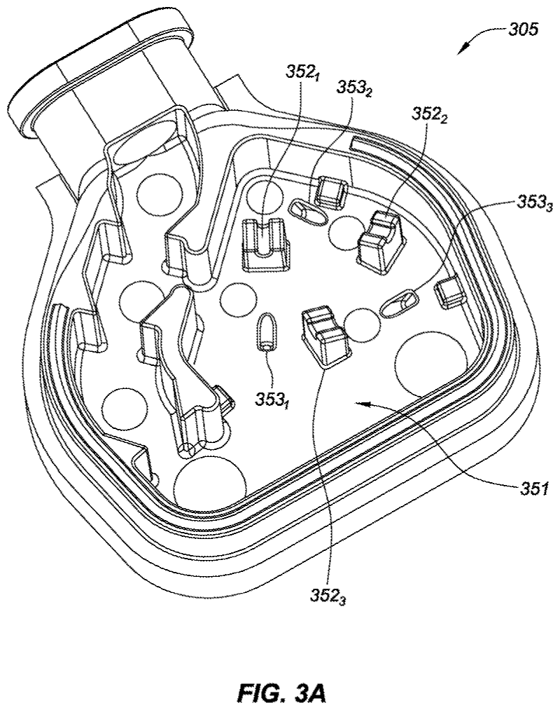

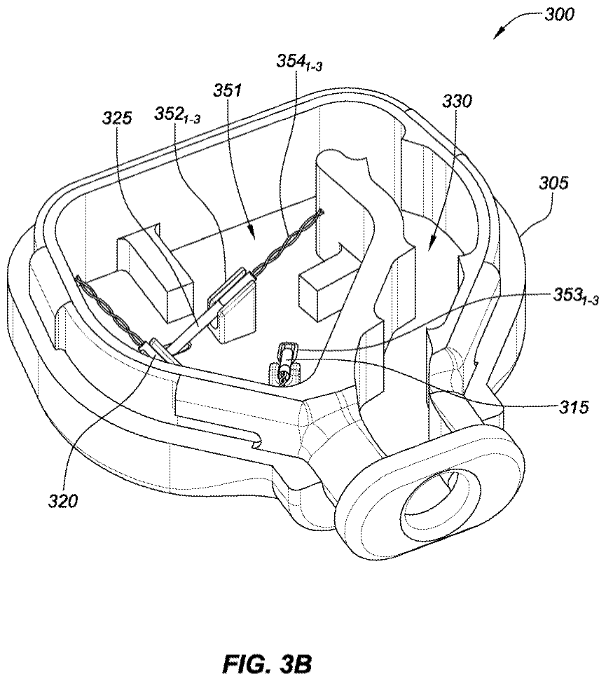

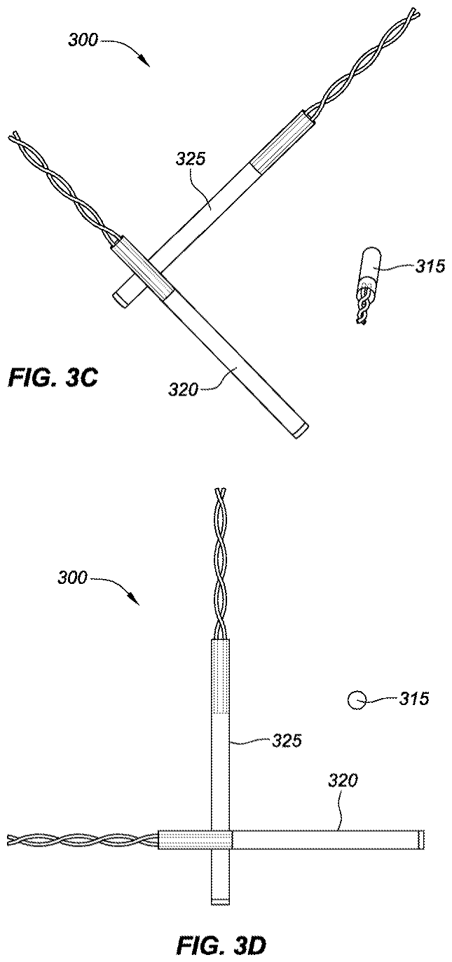

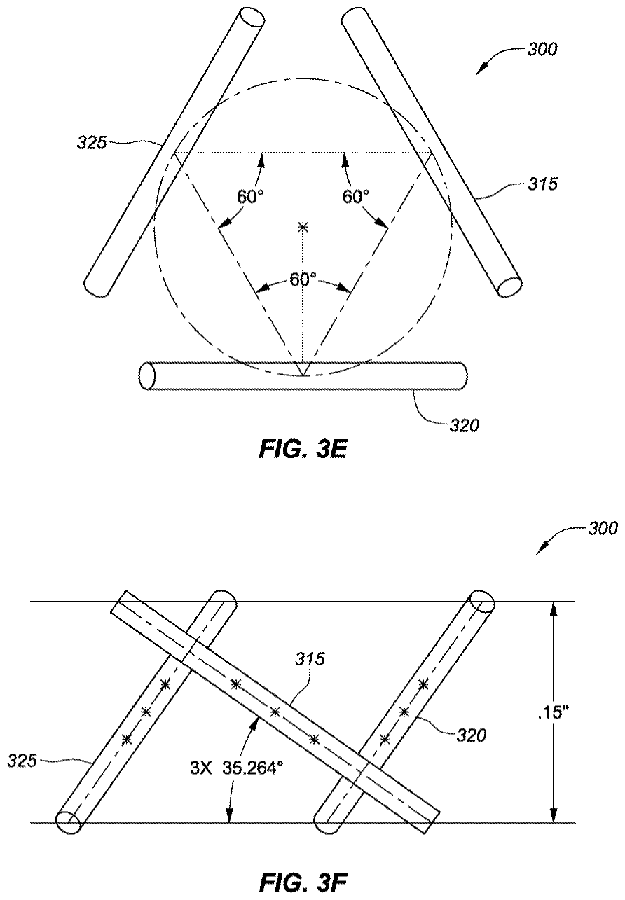

FIG. 2A is a simplified, isometric view of an exemplary catheter assembly 100 that can be used in conjunction with system 8. In some embodiments, and as shown in FIG. 2A, the catheter assembly 100 comprises a catheter 106 that includes a catheter tip assembly (also referred to as an electrode assembly or distal tip assembly) 112 at a distal end portion and operatively adapted for conducting a diagnostic or a therapeutic procedure under clinician control. A proximal end portion 104 of the catheter 106 may include a steering handle or other mechanism (not shown). In the present embodiment, catheter 106 is a mapping catheter. The catheter 106 includes a flexible shaft 102 extending between the proximal end portion 104 and the catheter tip assembly 112. The catheter assembly 100 further includes an electrical connector (not shown) configured to establish electrical connection(s) between the catheter tip assembly 112 and external electrical components (not shown) to perform, for example, localization, mapping, ablation, and/or pacing procedures. FIG. 2A further shows an introducer 114 comprising part of the catheter assembly 100. The catheter tip assembly 112 may comprise a plurality of sensors coils such as those shown, for example, in U.S. Pat. No. 6,690,963 (see, e.g., sensors 30, 32, 34 depicted in FIGS. 2 and 3), which has been incorporated herein by reference. These sensor coils may be located, for example, in the region shown by the dashed box 122 in FIG. 2B.

FIG. 2B is an enlarged, side view showing, in greater detail, the tip assembly 112. The tip assembly 112 includes a tip electrode 124, a plurality of ring electrodes 128_{R-2}, 128_{R-3}, and 128_{R-4}; and a plurality of electrical conductors 120 (e.g., one conductor electrically connected to each of the three ring electrodes and a separate conductor electrically connected to the tip electrode 124). Additional electrical connectors may extend proximally from the tip assembly 112 if sensor coils are located in, for example, the area outlined by dashed box 122. While the present embodiment discloses a mapping catheter, various types of catheters for both diagnostics and therapeutics may be utilized, and the respective magnetic distortion corrected position determined by the localization system disclosed herein.

FIG. 3A is an isometric top view of a magnetic reference sensor housing 305, consistent with various aspects of the present disclosure, with portions of the sensor housing broken away to reveal internal features. The sensor housing 305 of FIG. 3A houses three sensor coils via channels 352₁₋₃ and stops 353₁₋₃ (the stops extending into a surface 351 of the housing). For example, a first sensor coil may be placed into the channel 352₁, with a distal end of the sensor coil situation within stop 353₁. Once positively positioned within the channel and stop, a first sensor coil may be secured to the housing 305 using, for example, adhesive. The channels 352₂₋₃ and stops 353₂₋₃ similarly secure a second and third sensor coils. The stops 353₁₋₃ positively position the distal end of each of the sensor coils within a first plane, while the channels 352₁₋₃ positively position the proximal end of each of the sensor coils within a second plane. Further, the channels 352₁₋₃ and the stops 353₁₋₃ positively position the longitudinal axis of each of the sensor coils at right angles relative to one another.

FIG. 3B is an isometric top view of a magnetic reference sensor 300, consistent with various aspects of the present disclosure, with portions of a sensor housing 305 broken away to reveal internal features including a cavity 330 for housing sensor coils 315, 320 and 325. Each of the sensor coils are coupled to respective pairs of channels 352₁₋₃ and stops 353₁₋₃. As discussed above in reference to FIG. 3A, the channels and stops positively position the sensor coils 315, 320 and 325 within cavity 330. While various different embodiments for precisely positioning the sensor coils within the cavity are readily envisioned by a skilled artisan, the present embodiment utilizes channels and stops, and an adhesive application to secure the sensor coils to sensor housing 305. As shown in FIG. 3B, the stops 353₁₋₃ extend into a surface 351 of the sensor housing to facilitate enhanced placement of the sensor coils (e.g., manufacturing repeatability). The stops 353₁₋₃ positively position the distal end of each of the sensor coils 315, 320 and 325 within a first common plane, while the channels 352₁₋₃ positively position the proximal end of each of the sensor coils within a second common plane.

Lead wires 354₁₋₃ extend out from a proximal end of each of the sensor coils 315, 320 and 325 and may be routed through cavity 330 of sensor housing 305 before exiting the housing (in a common wiring bundle).

In some embodiments cavity 330 of sensor housing 305 may be potted to further prevent displacement of sensor coils 315, 320 and 325 relative to one another.

As discussed above, each of the sensor coils 315, 320 and 325 are affixed relative to sensor housing 305. In the present embodiment, the sensor coils are fixed in orthogonal orientations relative to one another. During operation, the magnetic reference sensor 300 is placed within a generated magnetic field and each of the respective sensor coils receive energy indicative of the strength and orientation of the magnetic field. In one specific embodiment, a vector sum of the received energy is computed to determine a perceived change in the position of the magnetic reference sensor due to patient respiration. However, where the magnetic reference sensor is exposed to eddy currents, a perceived change in the position of the magnetic reference sensor may actually be indicative of a magnetic distortion in the magnetic field proximal the magnetic reference sensor. In medical magnetic localization applications, such magnetic distortions affect the ability of the system to accurately locate a position of the magnetic reference sensor, and may cause the system to ignore the magnetic reference sensor entirely thereby disabling patient respiration tracking and correction.

Before transmitting the received signals from the first, second, and third sensor coils 315, 320, and 325, respectively, to computing circuitry for processing and determination of the amount of distortion in the magnetic field, sensor housing 305 may further house electronic circuitry which conducts a number of signal processing functions (e.g., analog-to-digital conversion, pre-amplification, and signal noise filtration). After signal processing, the received sensor coil signals are transmitted to computing circuitry of the magnetic localization system. In further embodiments, the magnetic reference sensor 300 may wirelessly transmit the received signals using wireless data transmission protocols known to one of skill in the art.

In the present embodiment, the sensor coil configuration facilitates six degree of freedom (6 DOF) determination of a magnetic reference sensor. Importantly, with the present configuration, only two sensor coils are necessary to calculate the 6 DOF. As such, the localization system may double-check or triple-check the 6 DOF determination of the magnetic reference sensor using any combination of two sensor coils.

FIG. 3C shows first, second, and third sensor coils, 315, 320, and 325 of the magnetic reference sensor 300, sans sensor housing 305. As shown in FIG. 3C, the sensor coils are oriented orthogonal to one another and affixed at precise distances from each other. During operation of the magnetic reference sensor in a magnetic field, the output of the sensor coil array is used to calculate the position and orientation of the magnetic reference sensor to track patient respiration and compensate catheter localization. When acceptable levels of magnetic field distortion at the magnetic reference sensor are exceeded, the localization system will disable respiration tracking/correction using the magnetic reference sensor. However, as all the sensor coils are configured non-parallel to many of the known sources of eddy currents within an electrophysiology catheter lab the sensor coils exhibit reduced sensitivity to eddy currents. As a result, the localization system may continue to operate respiration tracking/correction in many magnetically caustic environments.

FIG. 3C is a close-up, isometric top view of the magnetic reference sensor 300 of FIG. 3B, sans the sensor housing, and FIG. 3D is a close-up, isometric side view of the magnetic reference sensor of FIG. 3B, sans the magnetic reference sensor housing, consistent with various aspects of the present disclosure. FIGS. 3C and 3D further illustrate the perpendicularity of sensor coils 315, 320, and 325 of the magnetic reference sensor 300. In other words, each of the sensor coils are arranged orthogonal to one another. As a result, when a top/bottom surface of the magnetic reference sensor is aligned with a front facing surface of a magnetic field transmitter, a longitudinal axis associated with each of the sensor coils will have substantially equal non-perpendicularity to the front facing surface of the field transmitter. As a function of this substantially equal non-perpendicularity, all of the sensor coils have a non-vertical orientation.

FIG. 3E is a top view of the magnetic reference sensor 300 of FIG. 3B, sans the sensor housing, consistent with various aspects of the present disclosure. As shown in FIG. 3E, the angle of each sensor coil 315, 320, and 325 relative to one another is approximately 60°.

FIG. 3F is a side view of the magnetic reference sensor 300 of FIG. 3B, sans the sensor housing, consistent with various aspects of the present disclosure. The relative angle of each of the sensor coils 315, 320, and 325 relative to a horizontal surface (e.g., a surface 351 of the housing) is approximately 35°. Moreover, in the present embodiment, the vertical distance between a proximal end and a distal end of the sensor coils is 0.15 inches. In some specific embodiments, the angle is 35.264° (as shown in FIG. 3F).

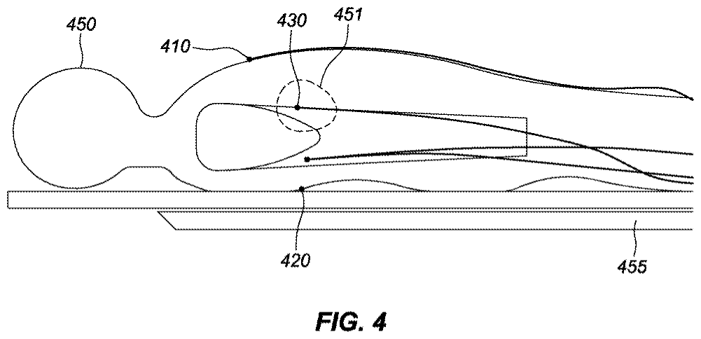

FIG. 4 shows an example application of a magnetic reference sensor 410 and a magnetic detection sensor 420 relative to a patient 450 during a medical procedure where magnetic localization of a medical device within a patient is utilized. In the present embodiment, the magnetic reference sensor 410 is placed anterior to the patient 450 (e.g., on a patient's chest), while the magnetic detection sensor 420 is placed posterior to the patient (e.g., between a patient's back and an operating table 455). In many example applications, the magnetic reference sensor and magnetic detection sensor are ideally located adjacent (and opposite one another) to the anatomy of the patient where the procedure is being conducted. As shown in FIG. 4, the sensors are opposite one another relative to the heart 451, which is receiving treatment by way of catheter 430, which is extended into the heart. One or more magnetic field transmitters adjacent the patient emit a magnetic field used to determine the position of the catheter. Specifically, the catheter, including one or more sensor coils in a distal tip region, senses the magnetic field in proximity to the one or more coils. Processing circuitry may then determine, based on the sensed magnetic field at the tip of the catheter, where the one or more coils are located in the magnetic field and, therefore, where the tip of the catheter is located. However, egress of other ferrous objects into the magnetic field create magnetic distortions within the field that affect localization of the catheter. Accordingly, the magnetic detection sensor 420 detects magnetic distortions in proximity to the posterior of the patient (e.g., medical instruments and equipment), and the magnetic reference sensor 410 detects patient respiration and other motion in proximity to the heart. In such a configuration, magnetic distortions and patient motion may be identified and a determination can be made as to the effect on the catheter.

In various embodiments of the present disclosure, to prevent inaccuracies in a magnetic localization system, the magnetic localization system may utilize one or more magnetic detection sensors to determine a variance between actual locations (based on the known/fixed position of the magnetic detection sensor within the system) and perceived locations (those determined based on the received magnetic fields at the magnetic detection sensor and post-processing). The determined variance is indicative of magnetic detection throughout the magnetic field due to egress of ferrous/metallic objects into the magnetic field. Based on the variance at each of the magnetic detection sensor locations, a transform may be computed to correct for the distortion at all locations within the magnetic field, including the magnetic distortion experienced by the medical device.

In various embodiments of a magnetic detection sensor, in accordance with the present disclosure especially in embodiments where fluoroscopy or other X-ray type imaging is required (during a medical procedure), the sensor may include materials that are transparent to X-ray imaging, and/or shaped to prevent interference with such imaging.

In various embodiments of the present disclosure, one or more magnetic detection sensors may be implemented in a hybrid-type localization system, such as the EnSite Precision™ Electro Anatomical Mapping System commercially available from St. Jude Medical, Inc. In such a configuration, impedance measuring patches are electrically coupled to the patient (e.g., on a patient's chest, on either side of patient's chest, and/or on at least one of patient's legs). Based on the varying impedance values detected by the impedance measuring patches, an impedance-based location of the medical device may be determined.

In various embodiments of the present disclosure, the magnetic detection sensor enables improved accuracy within a localization system by indicating when a location of the medical device provided by the magnetic field-based localization sub-system is inaccurate due to a magnetic distortion. In response, the mapping system may ignore the location data from the magnetic field-based portion of the system, or correct for the distortion using one or more of the methods disclosed herein. For example, data from one or more of the magnetic detection sensors may be used to determine a variance between actual locations (based on the known/fixed position of the magnetic detection sensor within the system) and perceived locations (those determined based on the received magnetic fields at the magnetic detection sensor and post-processing). The determined variance is indicative of magnetic distortion throughout the magnetic field due to egress of ferrous/metallic objects into the magnetic field. Based on the variance at each of the magnetic detection sensor locations, a transform may be computed to correct for the distortion at all locations within the magnetic field, including the magnetic distortion experienced by the medical device being magnetically localized.

In further more specific embodiments of the present disclosure, and consistent with all the above embodiments, magnetic detection sensors (and the sensor coils therein) may also be utilized to compensate for the sensed magnetic distortion. To compensate for magnetic distortion in the magnetic localization system, the fixed locations of the magnetic detection sensors provide a fixed reference frame. Based on the variance between the actual position (a known position or a position detected during calibration) and the perceived location of each of the magnetic detection sensors based upon the sensed magnetic field, the effect of the magnetic distortion throughout the magnetic field may be calculated and represented by a transform that restores the perceived locations of each of the magnetic detection sensors back to the respective actual positions. Similarly, the transform may be applied to the perceived location of a medical device within the magnetic field to determine a corrected (actual) location of the medical device.

Where an actual location of a magnetic detection sensor in a reference frame is not known (such as where the sensor is fixed to the patient), the relative location of the magnetic detection sensor to another magnetic detection sensor, where the distance between the two magnetic distortion sensors (or two or more coils within the same sensor) is fixed, can also be relied upon to correct for magnetic distortion in a magnetic localization system. The transform in such an embodiment being based upon a variance between the actual distances between the magnetic detection sensors and the perceived distances between the magnetic detection sensors based upon the sensed magnetic field at each of the magnetic detection sensors. The calculated transform may then be used to correct the perceived location of the medical device.

In embodiments such as those presented herein, the magnetic detection sensors are generally centered about the portion of the patient's body where the medical device localization is to take place. For example, in a cardiac-related operation, the magnetic detection sensors are desirably positioned in close proximity to the patient's heart to improve detection of magnetic distortions affecting the medical device therein.

In view of the present disclosure, various other configurations of a sensor for magnetic distortion detection and correction within a magnetic localization system for use during a medical procedure are readily envisioned. For example, in one embodiment, the sensor is positioned on a patient analog to replicate the positioning of a chest fixture and other leads/pads (e.g., electrocardiography (“ECG”) pads, impedance-based localization system pads, among others) on a patient for an intracardiac procedure. The sensor may be generally situated on a patient's chest in proximity to the area of the patient where magnetic localization is to take place (e.g., a target area). In more specific embodiments, a plurality of sensors encircle the target area to maximize magnetic distortion detection, which can emanate from a multitude of locations within the operating room.

Specific algorithms used in conjunction with the magnetic detection sensors and processing circuitry for determining the existence of magnetic distortions within the magnetic field and the effect of the magnetic distortion on magnetic localization of the catheter within the magnetic field are presented in U.S. application Ser. No. 15/416,059, filed 26 Jan. 2017, and is hereby incorporated by reference as though fully set forth herein.

This application incorporates by reference, as though fully set forth herein, U.S. application Ser. No. 15/416,059, filed 26 Jan. 2017.

It should be noted that the features illustrated in the drawings are not necessarily drawn to scale, and features of one embodiment may be employed with other embodiments as the skilled artisan would recognize, even if not explicitly stated herein. Descriptions of well-known components and processing techniques may be omitted so as to not unnecessarily obscure the embodiments of the disclosure. The examples used herein are intended merely to facilitate an understanding of ways in which the disclosure may be practiced and to further enable those of skill in the art to practice the embodiments of the disclosure. Accordingly, the examples and embodiments herein should not be construed as limiting the scope of the disclosure.

The terms “including,” “comprising” and variations thereof, as used in this disclosure, mean “including, but not limited to,” unless expressly specified otherwise.

The terms “a,” “an,” and “the,” as used in this disclosure, means “one or more,” unless expressly specified otherwise.

Although process steps, method steps, algorithms, or the like, may be described in a sequential order, such processes, methods and algorithms may be configured to work in alternate orders. In other words, any sequence or order of steps that may be described does not necessarily indicate a requirement that the steps be performed in that order. The steps of the processes, methods or algorithms described herein may be performed in any order practical. Further, some steps may be performed simultaneously.

When a single device or article is described herein, it will be readily apparent that more than one device or article may be used in place of a single device or article. Similarly, where more than one device or article is described herein, it will be readily apparent that a single device or article may be used in place of the more than one device or article. The functionality or the features of a device may be alternatively embodied by one or more other devices which are not explicitly described as having such functionality or features.

Various embodiments are described herein of various apparatuses, systems, and/or methods. Numerous specific details are set forth to provide a thorough understanding of the overall structure, function, manufacture, and use of the embodiments as described in the specification and illustrated in the accompanying drawings. It will be understood by those skilled in the art, however, that the embodiments may be practiced without such specific details. In other instances, well-known operations, components, and elements have not been described in detail so as not to obscure the embodiments described in the specification. Those of ordinary skill in the art will understand that the embodiments described and illustrated herein are non-limiting examples, and thus it can be appreciated that the specific structural and functional details disclosed herein may be representative and do not necessarily limit the scope of the embodiments, the scope of which is defined solely by the appended claims.

Reference throughout the specification to “various embodiments,” “some embodiments,” “one embodiment,” or “an embodiment,” or the like, means that a particular feature, structure, or characteristic described in connection with the embodiment is included in at least one embodiment. Thus, appearances of the phrases “in various embodiments,” “in some embodiments,” “in one embodiment,” or “in an embodiment,” or the like, in places throughout the specification are not necessarily all referring to the same embodiment. Furthermore, the particular features, structures, or characteristics may be combined in any suitable manner in one or more embodiments. Thus, the particular features, structures, or characteristics illustrated or described in connection with one embodiment may be combined, in whole or in part, with the features, structures, or characteristics of one or more other embodiments without limitation given that such combination is not illogical or non-functional.

It will be appreciated that the terms “proximal” and “distal” may be used throughout the specification with reference to a clinician manipulating one end of an instrument used to treat a patient. The term “proximal” refers to the portion of the instrument closest to the clinician and the term “distal” refers to the portion located furthest from the clinician. It will be further appreciated that for conciseness and clarity, spatial terms such as “vertical,” “horizontal,” “up,” and “down” may be used herein with respect to the illustrated embodiments. However, surgical instruments may be used in many orientations and positions, and these terms are not intended to be limiting and absolute.

What is claimed is:

1. A system to detect patient respiration in a magnetic field for localization of an intravascular catheter within a patient, the system comprising:
a magnetic field generator configured and arranged to generate the magnetic field for localization of the catheter within the patient;
a magnetic reference sensor including more than one sensor coil, each of the sensor coils including a longitudinal axis, and the sensor coils are configured and arranged to sense the magnetic field aligned with the longitudinal axis of the sensor coil and output an electrical signal indicative of the sensed magnetic field; and
wherein the magnetic reference sensor is positioned within the magnetic field generated by the magnetic field generator; and
wherein the longitudinal axis of each sensor coil is non-parallel relative to an axis of the generated magnetic field.

2. The system of claim 1, wherein the magnetic reference sensor includes three sensor coils.

3. The system of claim 1, wherein the longitudinal axis of each of the sensor coils are positioned substantially orthogonal relative to a longitudinal axis of the generated magnetic field.

4. The system of claim 2, wherein the three sensor coils are positioned substantially orthogonal relative to one another.

5. The system of claim 1, wherein each of the sensor coils include proximal and distal ends, each of the proximal ends of the sensors coils lie in a first common plane, and each of the distal ends of the sensors coils lie in a second common plane.

6. The system of claim 1, wherein each of the sensor coils are positioned within the magnetic reference sensor with a non-vertical orientation.

7. The system of claim 2, wherein signals from each of the three sensor coils are configured and arranged to determine six degrees of freedom of the magnetic reference sensor within the magnetic field.

8. The system of claim 2, wherein signals from any first pair of the three sensor coils are indicative of six degrees of freedom of the magnetic reference sensor, and a second pair of the three sensor coils verify the six degrees of freedom determined by the first pair.

9. The system of claim 2, wherein each of the three sensor coils are oriented 60° relative to one another about a vertical axis of the system.

10. The system of claim 1, wherein each of the sensor coils are oriented approximately 35° relative to one another about a horizontal axis of the system.

11. The system of claim 1, further including
processor circuitry communicatively coupled to the magnetic reference sensor, and configured and arranged to
receive electrical signals from the sensor coils indicative of the position of the magnetic reference sensor, and
detect a change in the position of the magnetic reference sensor over time associated with patient respiration.

12. The system of claim 1, wherein the sensor coils of the magnetic reference sensor are further configured and arranged to vary the electrical signal output in response to a change in the magnetic reference sensor position due to patient respiration.

13. The system of claim 11, wherein the processor circuitry is further configured to correct the calculated position of the catheter within the patient in response to the detected patient respiration.

14. An apparatus for detecting a position within a magnetic field, the apparatus comprising:
a first sensor coil;
a second sensor coil oriented orthogonal to the first sensor coil and fixedly positioned relative to the first sensor coil; and
a third sensor coil oriented orthogonal to the first and second sensor coils and fixedly positioned relative to the first and second sensor coils;
wherein the first, second, and third sensor coils are configured and arranged to sense a magnetic field strength and magnetic field orientation substantially coaxial with the respective sensor coil, each of the sensor coils positioned with substantially equal perpendicularity relative to a magnetic field axis.

15. The apparatus of claim 14, wherein each of the sensor coils are further configured and arranged to convert energy received from the magnetic field into an electrical signal, and variation of the electrical signal output of the sensor coil over time is indicative of a change in the position of the apparatus.

16. The apparatus of claim 14, wherein each of the sensor coils include proximal and distal ends, each of the proximal ends of the sensors coils lie in a first common plane, and each of the distal ends of the sensors coils lie in a second common plane.

17. The apparatus of claim 14, wherein each of the sensor coils have a non-vertical orientation.

18. The apparatus of claim 14, wherein each of the three sensor coils are oriented 60° relative to one another about a vertical axis of the system.

19. The apparatus of claim 18, wherein each of the sensor coils are oriented approximately 35° relative to one another about a horizontal axis of the system.
